(19) United States
(12) Patent Application Publication
(10) Pub. No.: US 2025/0152607 A1
(43) Pub. Date: May 15, 2025

(54) COMPOSITIONS FOR VITAMIN D PRODUCTION

(71) Applicant: DR WELLER LIMITED, Leatherhead (GB)

(72) Inventor: Michael FINNEN, Leatherhead (GB)

(21) Appl. No.: 18/838,640

(22) PCT Filed: Feb. 15, 2023
(86) PCT No.: PCT/GB2023/050339

§ 371 Date: Aug. 15, 2024

(30) Foreign Application Priority Data
Feb. 16, 2022 (GB) 2202031.7

(51) Int. Cl. A61K 31/575; A61K 8/14; A61K 8/19; A61K 8/44; A61K 8/58; A61K 8/63; A61K 9/127; A61K 31/198; A61K 31/685; A61K 33/00; A61K 45/06; A61Q 17/04

(52) U.S. Cl. CPC A61K 31/575; A61K 8/14; A61K 8/19; A61K 8/447; A61K 8/58; A61K 8/63; A61K 9/127; A61K 31/198; A61K 31/685; A61K 33/00; A61K 45/06; A61Q 17/04

(57) ABSTRACT

The present disclosure provides compositions which may be useful in the generation of vitamin D and/or nitric oxide, in particular when exposed to radiation (e.g. light and/or UV radiation). The disclosure further provides methods of generating vitamin D₂ and/or nitric oxide, and methods of treating and/or preventing conditions and/or diseases associated with and/or caused by a vitamin D and/or nitric oxide deficiency.

### FIELD

The present disclosure provides compositions which may be useful in the generation of vitamin D, in particular when exposed to radiation (e.g. light and/or UV radiation). The disclosure further provides methods of generating vitamin D₂, and methods of treating and/or preventing conditions and/or diseases associated with and/or caused by a vitamin D deficiency. The present disclosure further provides compositions which may find additional application in the generation of nitric oxide (NO), in particular when exposed to radiation (e.g. light and/or UV radiation). Accordingly, the disclosure further provides methods of generating nitric oxide, and methods of treating and/or preventing conditions and/or diseases associated with and/or caused by a NO deficiency and/or inhibited NO production.

### BACKGROUND

Vitamin D is known to be involved in the absorption of calcium, magnesium and phosphate; is important for the health of bones, teeth and muscles; and is known to play an important role in many other systems in the human body. A deficiency in vitamin D has been linked to many different diseases and conditions, including diseases of the skeletomuscular system (e.g. bone diseases such as Rickets, osteomalacia and osteoporosis), multiple sclerosis (MS), Alzheimer's Disease, cognitive impairment, asthma, cardiovascular disease, seasonal disorders, inflammation, depression diabetes, and cancer.

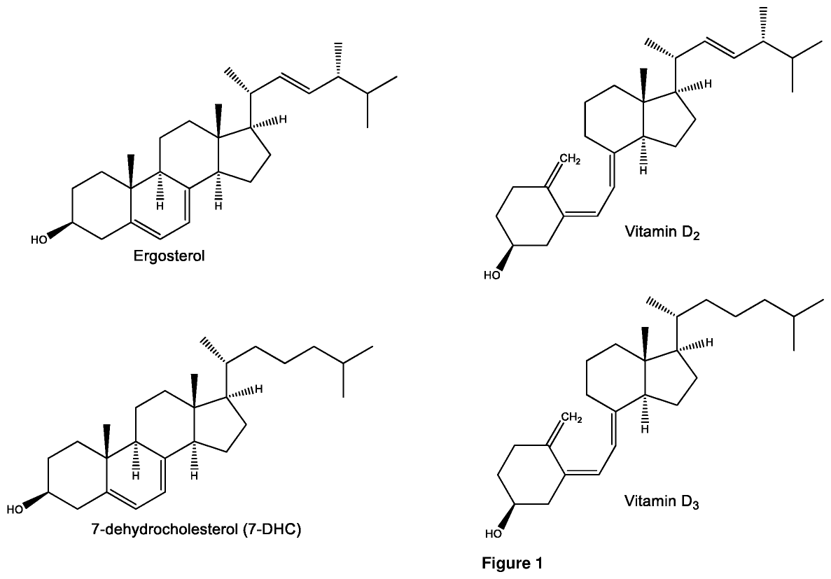

Vitamin D exists in several different forms, including vitamin D₂ (ergocalciferol) and vitamin D₃ (cholecalciferol) (see, for example, FIG. 1). Both vitamins D₂ and D₃ can be converted into active compounds via a two step enzymatic hydroxylation process in vivo. In the liver, vitamins D₂ and D₃ are converted to 25-hydroxyvitamin D. Subsequently, in the kidney, they converted into 1,25-dihydroxyvitamin D₂ or D₃ (calcitriol).

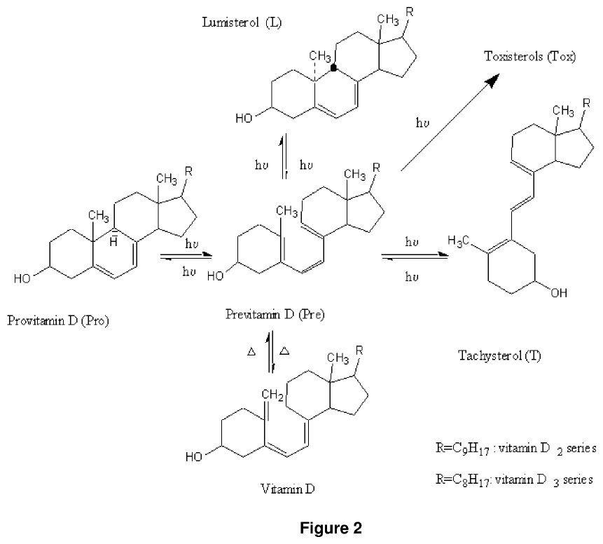

Vitamin D₃ is generated from vitamin D₃ precursors in the skin when exposed to UV light (in particular UV-B light). In particular, UV light induces the formation of pre-vitamin D₃ in the skin from 7-dehydro cholesterol (7-DHC), a compound which is naturally present in the skin. This then undergoes thermal re-arrangement to form vitamin D₃ (see, for example, FIG. 2) which then diffuses from the skin to the circulation and is converted to an active compound as described above. It follows that one of the most notable benefits of exposure to sunlight is its ability to boost your body's vitamin D supply.

An important factor in the production of vitamin D is the thermal rearrangement of pre-vitamin D to vitamin D and the role of membrane enhancement of the reaction in vivo. The isomerization rate of pre-vitamin D to vitamin D is relatively slow in an isotropic organic solvent such as hexane which at 37° C. takes up to 10 days to reach equilibrium (Xiao Q. Tian, Tai C. Chen, Lois Y. Matsuoka S, Jacobo Wortsman S, and Michael F. Holick, “Kinetic and Thermodynamic Studies of the Conversion of Previtamin D₃ to Vitamin D₃ in Human Skin”, THE JOURNAL OF BIOLOGICAL CHEMISTRY Vol. 268, No. 20, pp. 14888-14892, 1993). This contrasts with the in vivo situation where an increase in circulating vitamin D₃ is detected within 12 hours of UV irradiation. The rate of reaction for vitamin D₃ formation from pre-vitamin D₃ is believed to increase more than 10 fold by interaction with membrane phospholipids. Thus, the interactions between pre-vitamin D₃ and the surrounding biological environment have profound effects to the kinetics and thermodynamics of vitamin D₃ formation.

Additional model studies looking into the generation of vitamin D₃ are described in Holick et al, “A Liposomal Model that Mimics the Cutaneous Production of Vitamin D3”, The Journal of Biological Chemistry, 274, 1000, pages 4174-4179; and Holick et al, “Catalysed Thermal Isomerization between Previtamin D3 and Vitamin D3 via β-Cyclodextrin Complexation”, The Journal of Biological Chemistry, Vol. 270, 1995 and pages 8706 to 8711). Terentskaya et al have also looked at the photoisomerization of provitamin D in a hydrogel matrix (Terentskaya et al, Soft, 2013, 2, 8-12).

Ergosterol (a provitamin form of vitamin D₂) is a sterol found in fungi and protozoa and is also known to be converted to vitamin D₂ when exposed to UV light (see, for example, FIGS. 1 and 2). In some cases, ergosterol has been extracted and converted to vitamin D₂ for sale as a dietary supplement. Hungarian U.S. Pat. No. 102,939 describes a cosmetic composition (such as soap) comprising ergosterol which may be irradiated with UV radiation. Ergosterol powder is a known skin irritant.

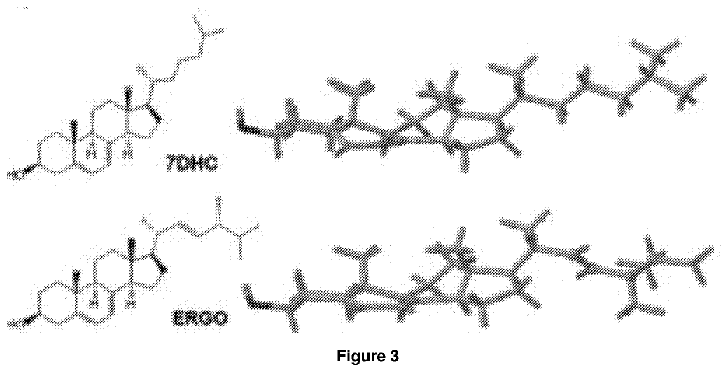

However, despite being structurally similar there are major differences between 7-DHC (provitamin D₃) and ergosterol (provitamin D₂) (see, for example, FIGS. 1 and 3) and the literature indicates that they differ particularly in their interaction with phospholipid membranes. By way of example, phosphatidylcholine (PC) liposomes containing ergosterol are fully solubilized in Triton X-100, whereas 7-DHC shows an increase in insolubility with increasing sterol content (Changfeng Chen and Carl P. Tripp, “A comparison of the behavior of cholesterol, 7-dehydrocholesterol and ergosterol in phospholipid membranes”, Biochimica et Biophysica Acta (BBA)—Biomembranes. Volume 1818, Issue 7, July 2012). Moreover, ergosterol incorporation into dipalmitoylphosphatidylcholine (DPPC) liposomes produces more domain formation than 7-DHC and major differences exist in their respective interactions with monounsaturated PC (Ya-Wei Hsueh, Mei-Ting Chen, Philipus J. Patty, Christian Code, John Cheng, Barbara J. Frisken, Martin Zuckermann and Jenifer Thewalt, “Ergosterol in POPC Membranes: Physical Properties and Comparison with Structurally Similar Sterols”, Biophysical Journal Volume 92 March 2007, 1606-1615). Differences in product formation following irradiation are also evident based on side chain length (Jianjun Chen, Andrzej T. Slominski, Duane D. Miller & Wei Li, “Effects of sidechain length and composition on the kinetic conversion and product distribution of vitamin D analogs determined by real-time NMR”, Dermato-Endocrinology, 2007, 5:1, 142-149).

It is well-documented that exposure to sunlight can also carry risks e.g. skin damage (sun burn/blistering), premature aging of the skin and skin cancer. The use of compositions to mitigate these risks (e.g. sunscreens and/or cosmetic compositions comprising agents having a sunlight protecting agent) is becoming increasingly widespread. As such, it is not uncommon for even those living in sunny climates to develop vitamin D deficiencies.

Some efforts have been made to address these problems. Holick et al describe topical compositions comprising lumisterol or tachysterol which photoisomerize to previtamin D₃ when exposed to low levels of UV radiation (WO 91/19479 and U.S. Pat. No. 5,167,953). Others have previously described compositions comprising vitamin D or cod liver oil (which comprises vitamin D) with sunscreens (see, for example, Person in US 2006/177390A1).

Alberts (U.S. Pat. No. 9,320,764B) described an alternative approach involving the use of an exposure indicator e.g. in the form of an adhesive skin patch. In this method, the topical administration of vitamin D is followed by administration of a sunscreen after the exposure indicator has changed state to indicate that the person has been exposed to sunlight for a sufficient amount of time.

Finnen (WO2018/042189) describes topical compositions (such as sunscreens) that comprise 7-dehydro cholesterol (7-DHC). These compositions were found to generate detectable levels of vitamin D₃ under UV radiation. Addition of nitric oxide (NO) generating components to the compositions greatly enhanced the production of vitamin D₃.

These methods have limitations. For example, vitamin D is currently banned from use in cosmetics in many territories (including the European Union and the US). In addition, 7-DHC is typically derived from animal sources and so is not suitable for use in products that are intended to be vegan or vegetarian-friendly.

Aside from the production of vitamin D, there are numerous health benefits that are thought to be associated with exposure to sunlight. For example, sunlight is known to have a positive effect on blood pressure (alleviating hypertension) and on a person's mood/wellbeing. Without wishing to be bound by theory, it has been suggested that at least some of the benefits associated with sunlight exposure require or result in nitric oxide (NO) production in the skin. Nitric oxide production in the skin has a number of therapeutic advantages; for example, it plays an important role in cellular signalling and is involved in many different processes. One of its key roles is as a short-lived but powerful vasodilator and it will be appreciate that this can have a direct and positive effect on blood pressure.

Thus, there remains a need to provide further compositions and methods to enhance and/or facilitate vitamin D and/or nitric oxide production in, or delivery to, a subject whilst also reducing the risk of excessive sunlight exposure.

### SUMMARY

The present disclosure is based on the identification that ergosterol can be used in certain compositions to facilitate, generate and/or enhance vitamin D production. In particular, the compositions comprising ergosterol as described herein can be applied topically to a subject to increase and/or augment levels of vitamin D (e.g. vitamin D₂) in the skin. In more particular examples, the present inventors have identified that one or more promoter agents may be added to the compositions to further augment, promote and/or enhance the generation of vitamin D. Some examples of these promoter agents may also augment, promote and/or enhance the generation of nitric oxide.

Indeed the present inventors have recognised that ergosterol may be topically applied to the skin of a subject and (following exposure of the subject to UV radiation) can be used to boost vitamin D (in particular vitamin D₂) levels in the subject. As such, this can be used to treat and/or prevent vitamin D deficiencies in a subject, and/or can be used to treat and/or prevent diseases or conditions associated with reduced levels of vitamin D.

Thus, according to a first aspect of the disclosure there is provided a composition for use in facilitating, augmenting and/or promoting the generation of vitamin D₂. The composition comprises ergosterol. The composition is formulated for topical administration.

In use, the composition may be applied or administered topically to a subject in need thereof. Upon exposure to sunlight (or another source of UV radiation), the ergosterol in the composition reacts and/or is converted into vitamin D₂ that may be delivered to the subject. In this way, the levels of vitamin D₂ in the subject can be augmented and/or increased.

In some examples, the described compositions can be those formulated, designed and/or intended to protect the skin from exposure to sunlight (and/or the UV component thereof) and/or which provide a degree of UV-protecting or sun-protecting effect. Surprisingly, it has been identified that even when formulated in such compositions ergosterol can still be converted to generate levels of vitamin D in the skin of a subject.

Thus, according to another aspect of the disclosure, there is provided a composition comprising:
(i) one or more UV-protecting and/or sunlight-protecting components; and
(ii) ergosterol.

In such compositions, it has been unexpectedly identified that ergosterol can generate levels of vitamin D₂ despite the presence of the one or more UV-protecting and/or sunlight protecting components in the composition.

The ergosterol that is used in these compositions may be derived from natural sources and/or may be prepared synthetically. In some examples, the ergosterol may be provided in and/or comprised within a natural extract.

As used herein, a “natural extract” may refer to an extract derived from a plant, animal, algal and/or microbial source. In particular, the natural extract comprising ergosterol may be derived from a fungal, algal or protozoa source (such as a fungal extract (e.g. a mushroom extract), an algal extract or a protozoa extract). Representative examples of fungal extracts (e.g. mushroom extracts) include, but are not limited to, Agaricus bisporus, Corthellus shitake, Gandoderma lucidium and Poria cocos. Representative examples of algal extracts include, but are not limited to, Gelidiella acerosa, Gigartina stellata, Hypnea musciformis and Laminaria cloustoni.

As used herein, an extract refers to a product that has been obtained via an extraction process performed on a source (e.g. a plant, algal or microbial source) as described herein. The extract may be solid or liquid. The extract may be obtained via an aqueous extraction process.

As used herein, the term “UV protecting component” may refer to an active component that functions to reduce the level of exposure (e.g. skin exposure) to UV radiation. For example, the active component may function to reduce the level of skin exposure to UV-A and/or UV-B radiation.

As used herein, the term “sunlight protecting component” may refer to an active component that functions to reduce the level of exposure (e.g. skin exposure) to sunlight (and optionally the UV, UV-A and/or UV-B components thereof).

The active components may take the form of organic chemicals, organic particulates, inorganic chemicals, and inorganic particulates. Some compositions may contain a plurality of different active components including, for example, combinations of different organic chemicals, organic particulates, inorganic chemicals, and inorganic particulates.

Suitable active components will be known to one of skill in this field. Such active components may reduce the level of exposure to sunlight and/or UV by absorbing the light (and/or UV component thereof). Additionally or alternatively, the active components may reduce the level of exposure to sunlight and/or UV by reflecting and/or scattering the light and/or UV component thereof.

An organic chemical active component may comprise a compound that absorbs UV light. An inorganic particulate active component may comprise a compound or moiety that reflects, scatters and/or absorbs light (including UV light). For example, silica, fumed silica, iron oxide, titanium dioxide and/or zinc oxide may be used.

An organic particulate active component may include those that absorb light (like the subset of useful organic chemicals) but which also contain multiple chromophores that (like the useful inorganic particulates) reflect and scatter a fraction of light. Tinosorb M is an example of a suitable organic particulate for use as an active component of a composition as described herein.

Representative examples of suitable active components include, but are not limited to, p-aminobenzoic acid (PABA), Padimate O (also known as OD-PABA or 2-ethylhexyl 4-(dimethylamino)benzoate), phenylbenzimidazole sulfonic acid, cinoxate (2-Ethoxyethyl p-methoxycinnamate), dioxybenzone (benzophenone-8), oxybenzone (benzophenone-3), homosalate (homomethyl salicylate, HMS), menthyl anthranilate (meradimate), octocrylene (2-cyano-3,3-diphenyl acrylic acid, 2-ethylhexylester), octyl methoxycinnamate (2-ethylhexyl-paramethoxycinnamate), octyl salicylate (2-Ethylhexyl salicylate), sulisobenzone (2-hydroxy-4-methoxybenzophenone-5-sulfonic acid, 3-Benzoyl-4-hydroxy-6-methoxybenzenesulfonic acid, trolamine salicylate (triethanolamine salicylate), avobenzone (1-(4-methoxyphenyl)-3-(4-tert-butyl phenyl) propane-1,3-dione), ecamsule (terephthalylidene dicamphor sulfonic acid), titanium dioxide, zinc oxide, 4-methylbenzylidene camphor, Parsol Max (Tinosorb M, Bisoctrizole, Methylene Bis-Benzotriazolyl Tetramethylbutylphenol, MBBT), Parsol Shield (Tinosorb S, Bis-ethylhexyloxyphenol methoxyphenol triazine, Bemotrizinol, BEMT, anisotriazine), Tinosorb A2B (Tris-Biphenyl Triazine), Neo Heliopan AP (bisdisulizole disodium, disodium phenyl dibenzimidazole tetrasulfonate, bisimidazylate, DPDT), Mexoryl XL (Drometrizole Trisiloxane), Benzophenone-9 (sodium dihydroxy dimethoxy disulfobenzophenone), Uvinul T 150 (octyl triazone, ethylhexyl triazone, EHT), Uvinul A Plus (diethylamino hydroxybenzoyl hexyl benzoate), Uvasorb HEB (iscotrizinol, diethylhexyl butamido triazone, DBT), Parsol SLX (dimethico-diethylbenzalmalonate, polysilicone-15), amiloxate (isopentyl-4-methoxycinnamate, isoamyl p-methoxycinnamate, IMC), and the like.

Thus, the compositions may comprise one or more active components, which are, when in use (and applied to the skin) designed to reduce (by, for example, light absorption and/or light reflecting/scattering based mechanisms) the exposure of skin to sunlight (and, for example, the UV-component thereof).

In some examples, it has been observed that the levels of vitamin D₂ generated from the compositions described herein are further increased when the one or more UV-protecting and/or sunlight-protecting components comprises an active component designed to reflect, scatter and/or absorb light (including UV light). Such compositions are sometimes referred to as “physical” sunscreens and may include, for example, silica, fumed silica, iron oxide, titanium dioxide and/or zinc oxide.

As used herein, a “physical sunscreen” is typically a composition comprising mineral particles that sit on the surface of the skin to reflect, absorb and/or scatter the UV radiation. Other types of sunscreen include those sometimes referred to as “chemical” or “organic” sunscreens. In these types of sunscreen, the active components may absorb into the skin, and then may act to absorb the UV radiation, convert this radiation to heat and release it from the body. Active components in chemical or organic sunscreens may include avobenzone, octinoxate, oxybenzone, octisalate, octocrylene, homosalate, octinoxate, or the like. Some sunscreens comprise a mixture of the different types of active component found typically in physical and chemical sunscreens.

The inventors have additionally identified that certain agents can be added to the compositions described herein to further promote and/or enhance vitamin D (e.g. vitamin D₂) production. In particular, the use of these promoter agents in the compositions of the disclosure can lead to an increased level of vitamin D₂ and/or can lead to an increased level of vitamin D₂ in the skin of a subject.

As such, according to a further aspect of the disclosure, there is provided a composition comprising:
(ii) ergosterol; and
(iii) one or more promoter agents to augment, promote and/or enhance the generation of vitamin D₂.

As stated above, the composition may further comprise: (i) one or more UV-protecting and/or sunlight-protecting components.

As used herein, the promoter agents may be any agent that is able to augment, promote and/or enhance the production of vitamin D₂. Without being bound by theory, it is hypothesised that, in some cases, these agents may facilitate and/or promote the generation of vitamin D₂ by complexing and/or interacting with ergosterol or a derivative thereof, to modulate the conversion of ergosterol to vitamin D₂.

Suitable promoter agents may include, but are not limited to, thiols, disulfides, NO-precursor compounds and phospholipids.

As stated above, suitable promoter agents may include liposomes, such as phospholipids. Without being bound by theory, the use of liposomes may induce conformational restraints due to amphipathic interactions with ergosterol or a derivative thereof, which may favour certain conformers of ergosterol (or of ergosterol derivatives) that ultimately may increase the rate of conversion to vitamin D₂ under UV radiation.

As used herein, the term “phospholipid” may refer to a class of lipids comprising a phosphate group and a plurality (e.g. two) fatty acids joined by an alcohol residue (which is typically a glycerol).

As used herein, a fatty acid may be a carboxylic acid with an aliphatic chain. The aliphatic chains of the fatty acids may be saturated or unsaturated (e.g. comprise one or more double or triple bonds) and/or may be straight, branched or contain cyclic structures. The fatty acids may each be independently selected from short-chain fatty acids (e.g. fatty acids with aliphatic chains containing between 1 and 5 carbon atoms), medium-chain fatty acids (e.g. fatty acids with aliphatic chains containing between 6 and 12 carbon atoms), long-chain fatty acids (fatty acids with aliphatic chains containing between 13 and 21 carbon atoms) and very long chain fatty acids (fatty acids containing above 22 carbon atoms, e.g. between 22 and 50 carbon atoms).

The phosphate group provides a hydrophilic head and the two fatty acids provide hydrophobic tail portions. Phospholipids may be amphiphilic and/or may have the ability to form lipid bilayers and/or micelles. In some examples, the phosphate group may be modified with additional organic molecules, such as choline, ethanoloamine or serine. In some examples, the phospholipid may be a phosphatidylcholine (such as one of those phosphatidylcholines described below).

Phospholipids may include those have a diacylglyceride structure such as phosphatidic acid (phosphatidate) (PA), phosphatidylethanolamine (cephalin) (PE), phosphatidylcholine (lecithin) (PC), phosphatidylserine (PS), phosphoinositides, phosphatidylinositol (PI), phosphatidylinositol phosphate (PIP), phosphatidylinositol bisphosphate (PIP2) and phosphatidylinositol trisphosphate (PIP3), and the like. Additional examples include sphingolipids such as ceramide phosphorylcholine (Sphingomyelin) (SPH), ceramide phosphorylethanolamine (Sphingomyelin) (Cer-PE), and ceramide phosphoryllipid, and the like.

Further representative examples include, but are not limited to saturated lipids, such as didecanoylphosphatidylcholine (DDPC), dilauroylphosphatidylcholine (DLPC), dipalmitoylphosphatidylcholine (DPPC), distearoylphosphatidylcholine (DSPC), diarachidoylphosphatidylcholine (DAPC) and unsaturated lipids, such as dipalmitoleoylphosphatidylcholine.

As stated above, other suitable promoter agents may include NO-precursor compounds, thiols and/or disulfides. For example, NO-precursor compounds, thiols and/or disulfides such as are described by Finnen (WO2018/042189) (the entire contents of which are incorporated herein by reference). Indeed the present inventors have observed that the addition of one or more of an NO-precursor compound, a thiol and a disulfide can enhance the production of vitamin D₂ in a sunscreen composition in response to UV irradiation. By way of further example, significant increases in vitamin D₂ production were observed following the addition of a thiol (e.g. glutathione) to an ergosterol-containing sunscreen composition. Yet more notable increases in vitamin D₂ production were observed following the addition of a thiol (e.g. glutathione) and a NO-precursor compound (e.g. a metal nitrate) to an ergosterol-containing sunscreen composition

Unexpectedly, and in addition to the beneficial effect on vitamin D₂ production, the present inventors have also identified that the combined use of ergosterol with an NO-precursor compound and a thiol or disulfide can enhance nitric oxide production from the NO-precursor compound and the thiol or disulfide compound.

Thus, according to a further example of the disclosure, there is provided a composition comprising:
(i) ergosterol;
(ii) a thiol compound and/or a disulfide; and
(iii) a NO-precursor compound.

Such a composition may additionally comprise one or more UV-protecting and/or sunlight-protecting components (as described herein). In other words, the composition may be a sunscreen composition.

In such examples, the combined use of ergosterol, an NO-precursor compound and a thiol or disulfide in a composition provides a synergistic combination, wherein not only does the NO-precursor compound and the thiol or disulfide enhance vitamin D₂ production (from ergosterol) but also does the ergosterol enhance nitric oxide production (from the nitrosothiol produced from the NO-precursor compound and thiol or disulfide compound). Together, such combinations of active ingredients can be used as additives for sunscreen compositions. Accordingly, the present disclosure further provides sunscreens which can mitigate or avoid the problem of prior art sunscreens which prevent the natural delivery of NO and vitamin D₂ via sunlight exposure.

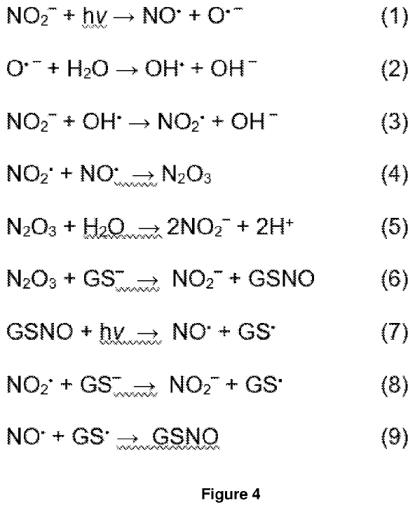

It has been previously identified that NO-precursor compounds when exposed to UV radiation in the presence of a thiol or disulfide compound, can be used to deliver nitric oxide. Without wishing to be bound by theory, the mechanism of UV-induced NO generation is illustrated by equations 1 to 5 in FIG. 4. Specifically, it is thought that UVA induced decomposition of nitrite (NO₂⁻) is self-limiting due to reaction with NO₂⁻ (see, for example, equation 4 in FIG. 4). However, in the presence of reduced thiols (e.g. reduced glutathione, denoted as GS⁻ in FIG. 4), nitrosothiols may be formed (see, for example equation 6, FIG. 4). The nitrosothiols may then undergo photolytic degradation to produce high levels of NO— (see equations 6-9 in FIG. 4). Furthermore, without being bound by theory, when the thiol or disulfide compounds described herein are exposed to radiation (such as UV radiation), they can undergo homolytic cleavage to form a thiyl radical (RS—). The newly-formed thiyl radical may trap NO— to form a nitrosothiol. Subsequently, the nitrosothiol may be photolytically cleaved to release NO— and regenerate the thiyl radical.

As stated, the generation of NO from the thiol or disulfide compounds described herein takes place following exposure to UV and in the presence of an NO-precursor. As such, when in use (and as further described below), any suitable thiol and/or disulfide compounds may be combined with any suitable NO-precursor compound.

As noted above, unexpectedly, the present inventors have also identified that the addition of ergosterol to a composition comprising an NO-precursor compound and a thiol or disulfide can enhance nitric oxide production from the NO-precursor compound, the thiol and disulfide.

Thus, as stated above, the present compositions as described according to various examples disclosed herein, may further comprise a NO-precursor compound.

By way of background, as used herein, a nitric oxide (NO)-precursor compound may be any species capable of reacting with the thiol or disulfide compound to generate a photolytically cleavable nitrosothiol compound. One or more NO-precursor compounds may be used together with any of the thiol and/or disulfide compounds described herein.

Representative examples include, but are not limited to, nitrite-containing compounds, nitrate-containing compounds and nitro-containing organic compounds.

For example, metal nitrites may be used as the NO-precursor compounds. Sodium nitrite (NaNO₂) is an example of one such NO-precursor compound. Other representative examples include, but are not limited to, potassium nitrite, dinitrosyl-iron complexes and other iron sulfur compounds etc.

In other cases, nitrate-containing compounds may be used, such as nitrate salts. Representative examples include, but are not limited to, thiamine nitrate and metal nitrates, such as sodium nitrate, potassium nitrate, silver nitrate, etc.

Additionally, or alternatively, the NO-precursor compound may be a nitro-containing organic group. For example, the NO-precursor compound may be a carboxylic acid comprising a nitro (—NO₂) group. Representative examples include, but are not limited to, nitropropionic acid and/or nitrooleate. For example, the NO-precursor compound may be 3-nitropropionic acid, 9-nitrooleate or 10-nitrooleate.

As stated above, the compositions according to the various examples described herein may further comprise a thiol and/or disulfide.

Without being bound by theory, the capacity of the thiol and/or disulfide compounds described herein to generate NO— in response to UV radiation may be influenced by a number of factors including, for example:
(i) the propensity of a thiol or disulfide compound to form the thiyl radical (RS—) when exposed to UV irradiation; and
(ii) the relative stability of the formed nitrosothiol to photolytic cleavage.

Thiol ionization may be understood to strongly influence the reactivity of the thiol group (e.g. the S—H bond of thiols dissociates with pKa approximately in the range 7-10). However, the formation of a thiyl radical is generally the result of a one-electron oxidation of thiols. For instance, in the processes described herein, the thiyl radical may be formed via a photolytic cleavage of a —S—H or —S—S-bond. In contrast to the chemical reactivity of the thiol group, it is believed that the response of thiols to UV radiation may be highly dependent on the groups adjacent to the thiol moiety. Similarly, it is hypothesised that the ability of disulfide bonds to form a thiyl radical (RS—) or dithiyl radical via a photolytic rupture of a disulfide (S—S) bond may be highly dependent on the individual chemical structures involved. It will be appreciated that a dithiyl radical may be generated after homolytic cleavage of a disulfide bond contained within a cyclic structure. Thus, the ability of any given thiol (RSH) and/or disulfide (RS—SR) compound to initiate a sustained NO generation process may depend on the individual chemical structures and groups present on the compound.

As such, the thiol or disulfide compounds described herein may comprise any compound capable of forming a photolytically cleavable nitrosothiol compound.

As used herein, the term photolytic cleavage may mean the homolytic rupture of a covalent bond to form two radical species upon exposure to radiation (e.g. UV radiation). This term may be used interchangeably with the terms photolysis and photolytic degradation.

Further, as used herein, the term thiol represents any compound comprising an —S—H moiety. In addition, as used herein, the term disulfide represents any compound comprising a —S—S-moiety, including persulfides (R—SSH). As will be appreciated, a disulfide compound may be considered as an oxidised form of a thiol compound. In addition, the various examples (methods, compositions, medicaments and the like) described herein may exploit a thiol compound in a reduced form (e.g. the thiolate anion, RS⁻).

The thiol or disulfide compound may be an organic compound comprising an —SH or —S—S— moiety respectively and also including persulfides (R—SSH). As used herein, an organic compound is a carbon-containing species that may optionally contain one or more heteroatoms such as N, O, S and/or P.

Accordingly, the thiol compound may be represented as:
(i) R—SH
(ii) the disulfide compound may be represented as R—S—S—R′ (where R and R′ may be identical groups or may be different);
(iii) the thiol compounds for use may comprise one of the following general formulae:

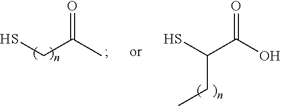

wherein n is from 0 (i.e. is absent) to any number up to 7.
(iv) the disulfide compounds for use may comprise one of the following general formulae:

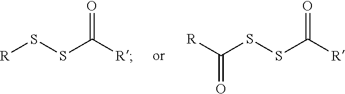

In each of the examples above, R and R′ may each independently be selected from: C₁-C₂₀, C₁-C₁₅, C₁-C₁₀, or C₁-C₅ alkyl group. These alkyl groups contain from 1 to 20, 1 to 15, 1 to 10 or from 1 to 5 carbon atoms respectively. As used herein, an alkyl group is selected from a straight, branched chain or cyclic hydrocarbon containing the defined number of carbon atoms. The alkyl group may be unsaturated (i.e. may comprise one or more double or triple bonds). Alternatively, R and R′ together may form a cyclic structure. For example, they may together form a 5-, 6- or 7-membered ring. The cyclic structure may optionally be substituted with one or more alkyl groups (as defined for R and R′ above).

The alkyl group may comprise substituents, optionally containing heteroatoms. For example, the alkyl group may be substituted with one or more carboxylic acid (—CO₂H), hydroxy (—OH) and/or amino (—NH₂) groups.

Optionally, one or more of the carbon atoms in the backbone of the alkyl chain may be replaced by a heteroatom containing functional group. For example, the alkyl chain may comprise one or more carbonyl, amide and/or ester groups. By way of further example, the alkyl chain may comprise a carbonyl group, such as a ketone. In some cases, the disulfide compound may be represented as

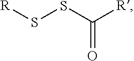

wherein R and R′ are as defined above.

In some examples, the thiol compound may comprise an amino acid or peptide comprising a thiol group. By way of example, the thiol compound may be a peptide comprising at least one —SH (thiol group), and comprising between about 2 and 10 amino acid residues, such as between about 2 and 5 amino acid residues. In some examples, the peptide may comprise about 3 amino acid residues (e.g. the peptide may be a tripeptide). The thiol compound may be a peptide (e.g. as described above) comprising at least one residue selected from cysteine and homocysteine. In some examples, the thiol compound may be glutathione.

Representative examples of the thiol compounds that can be used in the described compositions include, but are not limited to, glutathione, cysteine, homocysteine, cysteamine, and thiolactate. Exemplary structures are set out below:

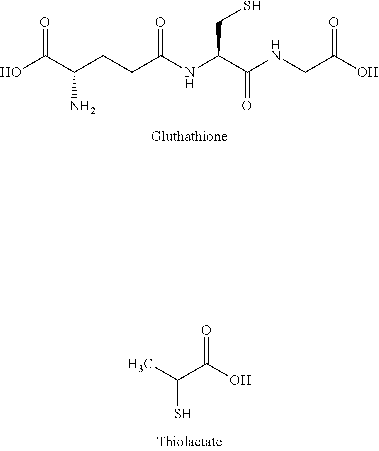

Whilst a particularly useful stereoisomer of glutathione has been illustrated above, it will be appreciated that other stereoisomers of glutathione (and any other thiol compounds—including those mentioned above) and/or, for example salts and derivatives thereof, may also be used in the methods and compositions of the disclosure. In particular, it should be noted that all salts, derivatives and/or sterioisomers should be functional—that is to say that they are capable of generating NO— in response to exposure to UV radiation (for example, in the presence of an NO-precursor compound).

Representative examples of disulfide compounds that can be used in the compositions of this disclosure include, but are not limited to, dithioglycolate, lipoic acid (oxidised) and cystine.

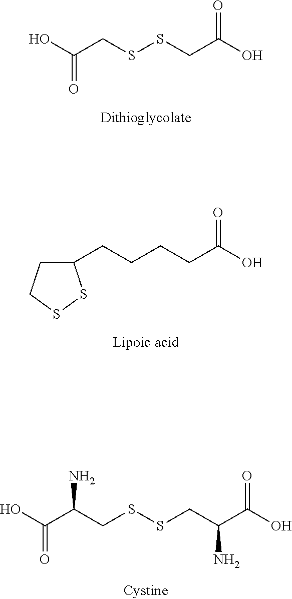 [SMILES: O=C(O)CSSCC(=O)O ; O=C(O)CCCCC1CCSS1 ; N[C@@H](CSSC[C@H](N)C(=O)O)C(=O)O]

Again, whilst a particularly useful stereoisomer of cystine has been shown above, it will be appreciated that other stereoisomers of cystine (and the other disulfide compounds) may also be used in the methods and compositions of the disclosure.

As stated above, the use of ergosterol in “physical sunscreen” compositions has been found to be particularly effective in generating vitamin D₂ (when the composition has been exposed to UV light and/or sunlight). As such, and without being bound by theory, the inventors hypothesise that the active components in these types of composition may act to augment, promote and/or enhance the production of vitamin D₂ from the ergosterol-containing composition. As such, suitable promoter agents may include an active component designed to reflect, scatter and/or absorb light (including UV light). Representative examples include, but are not limited to, fumed silica, and metal oxides (such as iron oxide, titanium dioxide and/or zinc oxide).

As stated above, the use of a promoter agent (such as a liposome or phospholipid, a NO-precursor compound and/or a thiol or disulfide compound) is able to augment, promote and/or enhance the production of vitamin D₂ from the ergosterol-containing composition. In some examples, it has been found that the addition of the promoter agent can provide at least a two-fold increase in the amount of vitamin D₂ produced from the composition (when compared to the amount of vitamin D₂ produced from the composition in the absence of the promoter agent). In some examples, the addition of the promoter agent can provide at least a three-fold, four-fold, five-fold, six-fold, seven-fold, eight-fold, nine-fold or ten-fold increase in the amount of vitamin D₂ produced from the composition (when compared to the amount of vitamin D₂ produced from the composition in the absence of the promoter agent).

Additionally or alternatively, the promoter agents may be cyclic oligosaccharides. As used herein, a cyclic oligosaccharide may refer to a polymer made up of a plurality of monosaccharide units arranged to form a ring structure (e.g. macrocylic ring structure). A class of particularly useful cyclic oligosaccharides may be cyclodextrins.

Without being bound by theory, cyclic oligosaccharides (such as cyclodextrin) may act to complex, interact and/or accommodate ergosterol to provide a microenvironment which favours the conversion of ergosterol to vitamin D₂.

As used herein, a “cyclodextrin” refers to a cyclic oligosaccharide comprising a macrocyclic ring of glucose subunits which are, e.g. joined by α-1,4-glycosidic bonds. Cyclodextrins may comprise from 5 to 40 glucose monomers.

Representative examples include, but are not limited to, α-cyclodextrin (e.g. comprising 6 glucose subunits), β-cyclodextrin (e.g. comprising 7 glucose subunits), γ-cyclodextrin (comprising 8 glucose subunits), and the like. In some cases, the cyclodextrin may comprise additional substituents e.g. methyl-β-cyclodextrin.

Additional agents that may be added to the compositions of the disclosure include hydrogels. In such cases, it has been identified that hydrogels (which are typically hydrophilic) do not adversely impact the conversion of the hydrophobic ergosterol to vitamin D₂.

Representative examples include, but are not limited to, hydrogels based on hydrophilic acrylamides, hydrophobic acrylonitrile and acrylic acids.

The compositions as described herein may comprise an amount of ergosterol that is able to provide a predetermined level of vitamin D₂ following exposure to UV radiation and/or sunlight. The exact amount of ergosterol to be added to and/or present in the composition may be dependent upon the level of UV radiation and/or intensity of the sunlight (or UV-component thereof). For example, relatively higher amounts of ergosterol may be added and/or present in compositions where the levels of UV radiation and/or intensity of the sunlight (or UV-component thereof) is expected to be lower. Additionally, or alternatively, the amount of ergosterol to be added to and/or present in the composition may be dependent upon the desired amount of vitamin D₂ to be provided. For example, if a higher amount of vitamin D₂ is desired, a higher amount of ergosterol may be added to and/or be present in the composition.

In some examples, the composition may comprise between about 0.0001 wt % and about 25 wt % of ergosterol based on the total weight of the composition. In some examples, the composition may comprise between about 0.001 wt % and about 10 wt % of ergosterol based on the total weight of the composition. In some examples, the composition may comprise between about 0.005 wt % and about 5 wt % of ergosterol based on the total weight of the composition. In some examples, the composition may comprise between about 0.0075 wt % and about 1 wt % or about 0.1 wt % (such as approximately 0.01 wt %) of ergosterol based on the total weight of the composition.

Where the promoter agents are present in the composition, they may be used in an amount that augments, promotes and/or enhances the production of vitamin D₂ to provide a desired or target amount of vitamin D₂.

By way of example (and without being bound by theory), where the promoter agents comprise liposomes (such as phospholipids), the molar ratio of the ergosterol to the promoter agent may be such that the phospholipids are able to form micellar arrangements (e.g. micelles) around the ergosterol.

In some examples, the molar ratio of the ergosterol to the phospholipid is between about 1:10 and 1:200, or between about 1:25 and 1:100. In some examples, the molar ratio of the ergosterol to the promoter agent (e.g. phospholipid) may be approximately 1:50.

Where a liposome (e.g. a phospholipid) is present in the composition, the composition may comprise between about 0.0001 wt % and about 1 wt % or between about 0.001 wt % and about 0.5 wt % of the liposome (e.g. a phospholipid) based on the total weight of the composition.

Where an NO-precursor compound is present in the composition, the composition may comprise between about 0.0001 wt % and about 10 wt % of the NO-precursor compound based on the total weight of the composition. In some examples, the composition may comprise between about 0.001 wt % and about 5 wt % of the NO-precursor compound based on the total weight of the composition. In some examples, the composition may comprise between about 0.005 wt % and about 2.5 wt % of the NO-precursor compound based on the total weight of the composition. In some examples, the composition may comprise between about 0.01 wt % and about 1 wt %, or between about 0.05 wt % and about 0.5 wt %, or between about 0.075 wt % and about 0.2 wt % (such as approximately 0.1 wt %) of the NO-precursor compound based on the total weight of the composition.

Where a thiol or disulfide is present in the composition, the composition may comprise between about 0.0001 wt % and about 10 wt % of the thiol or disulfide compound based on the total weight of the composition. In some examples, the composition may comprise between about 0.001 wt % and about 5 wt % of the thiol or disulfide compound based on the total weight of the composition. In some examples, the composition may comprise between about 0.005 wt % and about 2.5 wt % of the thiol or disulfide compound based on the total weight of the composition. In some examples, the composition may comprise between about 0.01 wt % and about 1 wt %, or between about 0.05 wt % and about 0.5 wt %, or between about 0.075 wt % and about 0.2 wt % (such as approximately 0.1 wt %) of the thiol or disulfide compound based on the total weight of the composition.

By way of further example, an exemplary composition as described herein may comprise:
(i) ergosterol in an amount between about 0.0075 wt % and about 1 wt % (based on the total weight of the composition);
(ii) a thiol compound and/or a disulfide (e.g. glutathione) in an amount between about 0.05 wt % and about 0.5 wt % (based on the total weight of the composition); and
(iii) a NO-precursor compound (e.g. a metal nitrate) in an amount between about 0.05 wt % and about 0.5 wt % (based on the total weight of the composition); and optionally wherein the composition comprises:
(iv) a liposome (e.g. a phospholipid) in an amount between about 0.001 wt % and about 0.5 wt % (based on a total weight of the composition).

As stated previously, such a composition may additionally comprise one or more UV-protecting and/or sunlight-protecting components (as described herein).

Further details relating to the compositions described herein and their uses are now provided.

The compositions disclosed herein may be suitable for topical application. For example, the compositions may be formulated for topical application. The compositions may be topically applied to any part of the skin (and also hair and nails), including skin that would typically be exposed to the sun when a subject is outside (such as the face, hands, neck, arms, legs, ears, shoulders, feet, torso, lips etc).

The compositions may be provided in the form of a cream, liquid, ointment, serum, oil, foam, scrub, gel, toner or the like.

The compositions of the disclosure be formulated together with pharmaceutically, therapeutically and/or cosmetically acceptable diluents, excipients, carriers and the like. For example, a composition according to the present disclosure may be prepared conventionally, comprising substances that are customarily used in, for example, pharmaceutical compositions and as described in, for example, Remington's The Sciences and Practice of Pharmacy, 22nd Edition (Pharmaceutical Press 2012) and/or Handbook of Pharmaceutical Excipients, 7th edition (compiled by Rowe et al, Pharmaceutical Press, 2012)—the entire content of all of these documents and references being incorporated by reference.

In some examples, the compositions may be formulated with suitable stabilizers, wetting agents, emulsifiers, salts (for use in influencing osmotic pressure), buffers and/or other substances that do not react deleteriously with the active compounds (e.g. ergosterol). The compositions may comprise diluents other than water including, for example, liquid or solid emollients, solvents, humectants, thickeners and powders.

A composition of the disclosure may comprise or further comprise cosmetically acceptable excipients and/or bases. Suitable excipients and/or bases may have a cream, lotion, gel or emulsion format. Suitable bases may include creams commonly known as “vanishing creams” and which comprise an amount of fatty acid (for example 3 to 25%, more preferably 5 to 20% fatty acid) and optionally “soaps” (which may include alkali metal salts of fatty acids, like sodium or potassium salts) and water.

Compositions of this disclosure may be cosmetic compositions. The composition may be selected from the group consisting of moisturisers, skin conditioning agents, make-up (e.g. foundation, lipstick) and the like.

As stated above, the composition may be one that is formulated, designed and/or intended to protect the skin from exposure to sunlight (and/or the UV component thereof) and/or to provide a degree of UV-protecting or sun-protecting effect.

By way of example only, the composition may be a sunscreen. As used herein, a sunscreen includes those compositions referred to as, for example, “masks”, “sunblock”, “suntan lotion”, “sun cream” and certain types of “after sun”.

As indicated above, the composition may be a cosmetic composition that when applied to the skin has a UV or sunlight protecting effect. Indeed, the composition may be any product with an SPF rating. In addition to those non-limiting examples already mentioned, other non-limiting examples may include antiperspirants, deodorants, lipsticks, chap-sticks, foundations, mascara, sunless tanners and fake-tan compositions.

Compositions which are designed to reduce the exposure of skin to sunlight are generally provided in (or with) a variety of sun protection factor (SPF) ratings. The SPF rating is a measure of the fraction of sunburn-producing UV sunlight rays which reach the skin. The higher the SPF rating, the fewer rays reach the skin. Thus, a composition with an SPF rating of 15 will allow more of the sun's UV rays to reach the skin than a composition which is SPF rated 50. Ergosterol may be used to facilitate, generate and/or enhance vitamin D (e.g. vitamin D₂) production in compositions with an SPF rating of at least 5, at least 10, at least 15, at least 20, at least 25, at least 30, at least 35, at least 40 or at least 50.

There is further provided a method for increasing and/or augmenting levels of vitamin D (e.g. vitamin D₂) in a subject in need thereof, said method comprising using, applying and/or administering ergosterol (or a composition as described herein) to the subject (and, in particular, to the skin of the subject).

In such a method, the application or administration of ergosterol or the composition to the skin of said subject is such that when the subject is exposed to a source of UV light (including the sun), ergosterol will react and/or be converted to generate vitamin D₂ and/or deliver vitamin D₂ to the subject. As such, the method may further comprise exposing the subject (e.g. at least a part of the skin of a subject) to UV radiation (e.g. sunlight or the UV component thereof).

According to a yet further aspect of the disclosure, there is provided a method for treating and/or preventing diseases or conditions associated with reduced levels of vitamin D (e.g. vitamin D₂) and/or a vitamin D (e.g. vitamin D₂) deficiency, said method comprising administering to a subject in need thereof a therapeutically effective amount of a composition as disclosed herein. In particular, the method may comprise topically administering to the subject in need thereof the therapeutically effective amount of a composition as disclosed herein.

In some examples of the disclosure, there is provided a method for treating and/or preventing diseases or conditions associated with reduced levels of vitamin D (e.g. vitamin D₂) and/or a vitamin D (e.g. vitamin D₂) deficiency, said method comprising topically administering to a subject in need thereof a therapeutically effective amount of ergosterol.

Again, the application or administration of ergosterol and/or the composition to the skin of said subject is such that when the subject is exposed to a source of UV light (including the sun), ergosterol will react and/or be converted to generate vitamin D₂ and/or deliver vitamin D₂ to the subject. As such, the method may further comprise exposing and/or subjecting the subject (e.g. or at least a part of the skin of a subject) to UV radiation (e.g. sunlight or the UV component thereof).

There is further provided a composition (as disclosed herein) for use in a method for treating and/or preventing diseases or conditions associated with reduced levels of vitamin D and/or a vitamin D deficiency, said method comprising administering (e.g. topically administering) to a subject in need thereof a therapeutically effective amount of a composition as disclosed herein.

In other examples, there is provided ergosterol for use in a method for treating and/or preventing diseases or conditions associated with reduced levels of vitamin D and/or a vitamin D deficiency, said method comprising topically administering to a subject in need thereof a therapeutically effective amount of ergosterol.

Again, the application or administration of ergosterol or the composition to the skin of said subject is such that when the subject is exposed to a source of UV light (including the sun), ergosterol will react and/or be converted to generate vitamin D₂ and/or deliver vitamin D₂ to the subject. As such, the method may further comprise exposing and/or subjecting the subject (e.g. or at least a part of the skin of a subject) to UV radiation (e.g. sunlight or the UV component thereof).

Additionally or alternatively, there is provided a use of ergosterol or a composition (as disclosed herein) in the manufacture of a medicament for use in:
(i) the treatment and/or prevention of diseases or conditions associated with reduced levels of vitamin D and/or a vitamin D deficiency; and/or
(ii) a method for increasing and/or augmenting levels of vitamin D (e.g. vitamin D₂) in a subject in need thereof.

As used herein, diseases or conditions associated with reduced levels of vitamin D and/or a vitamin D deficiency include, but are not limited to, diseases of the skeletomuscular system (e.g. bone diseases such as Rickets, osteomalacia and osteoporosis), multiple sclerosis (MS), Alzheimer's Disease, cognitive impairment, asthma, cardiovascular disease, seasonal disorders, inflammation, depression diabetes, and cancer.

As stated above, where ergosterol is used in compositions comprising an NO-precursor compound and a thiol or disulfide, it has unexpectedly been observed that the addition of the ergosterol also leads to an enhanced nitric oxide production.

Thus, there is further provided a method for increasing and/or augmenting levels of nitric oxide (e.g. and also levels of vitamin D) in a subject in need thereof, said method comprising using, applying and/or administering a composition comprising ergosterol, an NO-precursor compound and a thiol or disulfide (as described herein) to the subject (and, in particular, to the skin of the subject).

According to a yet further aspect of the disclosure, there is provided a method for treating and/or preventing diseases or conditions associated with reduced levels of nitric oxide and/or nitric oxide deficiency, said method comprising administering to a subject in need thereof a therapeutically effective amount of a composition as disclosed herein (e.g. a composition comprising ergosterol, the NO-precursor compound, and the thiol or disulfide compound). In particular, the method may comprise topically administering to the subject in need thereof the therapeutically effective amount of a composition as disclosed herein.

In such methods, the application or administration of the composition to the skin of said subject is such that when the subject is exposed to a source of UV light (including the sun), the NO-precursor compounds in the presence of a thiol or disulfide compound will react to generate and/or deliver nitric oxide (NO.) to the subject. As such, the methods may further comprise exposing and/or subjecting the subject (e.g. or at least a part of the skin of a subject) to UV radiation (e.g. sunlight or the UV component thereof).

There is further provided a composition (as disclosed herein) for use in a method for treating and/or preventing diseases or conditions associated with reduced levels of nitric oxide and/or a nitric oxide deficiency (e.g. as well as diseases or conditions associated with reduced levels of vitamin D and/or a vitamin D deficiency), said method comprising administering (e.g. topically administering) to a subject in need thereof a therapeutically effective amount of a composition as disclosed herein and for example, comprising ergosterol, the NO-precursor compound, and the thiol or disulfide compound. Again, the method may further comprise exposing and/or subjecting the subject (e.g. or at least a part of the skin of a subject) to UV radiation (e.g. sunlight or the UV component thereof).

Additionally or alternatively, there is provided a use of a composition comprising ergosterol, the NO-precursor compound, and the thiol or disulfide compound (as disclosed herein) in the manufacture of a medicament for use in:
(i) the treatment and/or prevention of diseases or conditions associated with reduced levels of nitric oxide and/or a nitric oxide deficiency; and/or
(ii) a method for increasing and/or augmenting levels of nitric oxide in a subject in need thereof.

As used herein, diseases or conditions associated with reduced levels of nitric oxide and/or a nitric oxide deficiency include, but are not limited to, diseases and/or conditions which affect, for example the cardiovascular system such as hypertension, (nitric oxide being important in regulating circulation and dilation of blood vessels of all types), the respiratory tract (e.g. via effects on blood circulation), the musculoskeletal system (e.g. via effects on muscle oxygenation and the like), cellular function (e.g. via effects on blood vessel development), the immune system, the nervous system, gastrointestinal tract and the urogenital system, (including matters connected with sexual health such as erectile dysfunction).

By way of further representative example, diseases or conditions associated with reduced levels of nitric oxide and/or a nitric oxide deficiency include (but are not limited to) hypertension and renal damage (see, for example, Baylis et al, Curr. Opin. Nephrol. Hypertens., 1996, 5(1): 80-88); mitochondrial disorders (see, for example, Almanni et al, Front. Mol. Neurosci. Volume 14, 2021, Article 682780); sexual dysfunction e.g. erectile dysfunction and vasculogenic female sexual dysfunction; chronic kidney disease; congenital abnormalities including achalasia cardia, hypertrophic pyloric stenosis, and Hirschsprung disease; Parkinson's Disease, Alzheimer's Disease, amyotrophic lateral sclerosis (ALS), Huntingdon's disease and ischemic brain injury (see, for example, Knott et al, Antioxid Redox Signal. 2009 March; 11(3): 541-553: nitric oxide has been implicated as a mediator in neurodegeneration in such diseases).

In particular, the compositions, methods and uses described herein may be of particular utility as a prophylactic treatment to prevent the occurrence of conditions or diseases associated with reduced levels of vitamin D and/or a vitamin D deficiency, and/or conditions or diseases associated with reduced levels of nitric oxide and/or a nitric oxide deficiency.

In any of the compositions, methods and uses described herein, the ergosterol and/or composition may be applied topically to the subject in need thereof. The ergosterol and/or compositions may be applied or administered to the skin of the subject or at least part of the skin of subject. In particular, the ergosterol and/or compositions may be applied to areas of skin of the subject that are most likely to be exposed to the sunlight (or UV component thereof), such as the face, hands, neck, arms, legs and/or feet etc.

The composition may be administered, applied or used by a subject who is to be exposed to the sun and/or a subject susceptible, predisposed to or at risk of developing a disease or condition associated with reduced levels of vitamin D and/or a vitamin D deficiency, and/or associated with reduced levels of nitric oxide and/or a nitric oxide deficiency.

In some examples, the subject may be one who is susceptible, predisposed to or at risk of developing a disease or condition associated with reduced levels of vitamin D and/or a vitamin D deficiency (and/or associated with reduced levels of nitric oxide and/or a nitric oxide deficiency) due to their geographic location and/or their lifestyle. For example, the subject may be one who lives in an area of lower levels of sunlight (such as the northern hemisphere). In some examples, the subject may be one who is exposed to lower levels of sunlight due to a working pattern (e.g. night shift workers). In other examples, the subject may be one who is susceptible, predisposed to or at risk of developing a disease or condition associated with reduced levels of vitamin D and/or a vitamin D deficiency (and/or associated with reduced levels of nitric oxide and/or a nitric oxide deficiency) due to a genetic predisposition (e.g. due to skin tone). For example, the compositions of the disclosure may be of use to augment, increase and/or enhance vitamin D (and/or nitric oxide) in individuals with darker skin pigmentation. In other examples, the subject may be one who lives in an area of relatively high sunlight but who may be one who is susceptible, predisposed to or at risk of developing a disease or condition associated with reduced levels of vitamin D and/or a vitamin D deficiency (and/or associated with reduced levels of nitric oxide and/or a nitric oxide deficiency) due to regular use of sunscreen compositions.

The compositions as described herein may be administered to, applied to and/or used by a subject prior to sunlight exposure and/or prior to exposure to UV radiation. For example, a subject may apply or administer the compositions described herein to any area of skin that will be exposed to sunlight prior to going outside. The compositions may be used for extended periods of time and may be administered to, applied to and/or used by a subject regularly (e.g. hourly, daily or weekly basis, etc.).

There is further provided a method for preparing a composition as described herein. The method may comprise adding ergosterol to one or more UV-protecting and/or sunlight-protecting components to provide the composition. Alternatively or additionally, the method may further comprise formulating the composition with one or more pharmaceutically, therapeutically and/or cosmetically acceptable diluents, excipients or carriers. The method may comprise formulating the ergosterol and/or the composition such that it is suitable for topical administration.

The method may further comprise adding one or more promoter agents (as described herein) to provide the composition. Additionally or alternatively, the method may comprise adding a hydrogel to the composition.

In some examples, ergosterol and the one or more promoter agents may be formulated together prior to addition to the composition. In such cases, the composition may be prepared by providing a composition suitable for topical administration and/or a composition comprising one or more UV-protecting and/or sunlight-protecting components (optionally together with one or more pharmaceutically, therapeutically and/or cosmetically acceptable diluents, excipients or carriers) and supplementing the composition with a formulation comprising the ergosterol and the one or more promoter agents.

In some examples, where the promoter agent is, or comprises, a liposome (such as a phospholipid), the ergosterol may be incorporated into the liposome (e.g. the phospholipid) prior to addition to the composition. For example, the method may comprise forming liposome (e.g. phospholipid) micelles that encapsulate, incorporate and/or comprise the ergosterol prior to addition to the composition.

It should be understood that throughout this specification, the terms “comprise”, “comprising” and/or “comprises” is/are used to denote that aspects and embodiments of this disclosure “comprise” a particular feature or features. It should be understood that this/these terms may also encompass aspects and/or embodiments which “consist essentially of” or “consist of” the relevant feature or features.

### DETAILED DESCRIPTION

The present disclosure will now be described in detail (and by way of example only) by reference to the following figures which show:

FIG. 1 shows the chemical structures for ergosterol, 7-dehydrocholesterol, vitamin D₂ and vitamin D₃ respectively.

FIG. 2 shows an overview of the various chemical reactions involved in the conversion of ergosterol and 7-dehydrocholesterol to vitamin D₂ and D₃ respectively.

FIG. 3 shows a comparison of the chemical structures of 7-DHC and ergosterol (“ERGO”).

FIG. 4 shows the mechanism of UV-induced nitric oxide (NO) generation in the presence of an NO-precursor compound and a thiol or disulfide compound.

### EXPERIMENTAL SECTION

### Sunscreen Formulations

Details of the sunscreen formulations used in the following investigations (in particular, Examples 1 to 4) are detailed below.

Exemplary oil-in-water sunscreen formulations of the ingredients listed in Tables 1 and 2 were made according to standard procedures. Titanium dioxide and Zinc oxide were used as physical sun filters. Homosalate, Octocrylene, Ethylhexyl Salicylate and Ethylhexyl Methoxycinnamate were used as organic sun filters.

| PHYSICAL SUNSCREEN INCI¹ List | % concentration in bulk (w/w) |
|---|---|
| Aqua | 50.08 |
| Isohexadecane | 9 |
| Propylheptyl Caprylate | 9 |
| Cyclopentasiloxane | 6 |
| Titanium Dioxide | 4 |
| Zinc Oxide | 4 |
| C12-15 alkyl benzoate | 3.84 |
| Triethylhexanoin | 3.6 |
| Polyglyceryl-3 Diisostearate | 3 |
| Euphorbia Cerifera | 2.5 |
| Glycerin | 2.5 |
| Magnesium Sulphate Heptahydrate | 1 |
| Aluminium Stearate | 0.24 |
| Alumina | 0.16 |
| Polyhydroxystearic acid | 0.08 |
| Phenoxyethanol | 0.9 |
| Ethylhexylglycerin | 0.1 |

¹INCI: International Nomenclature Cosmetic Ingredients.

Table 1 showing the composition of an exemplary physical sunscreen composition (“Physical Sunscreen A”).

| ORGANIC SUNSCREEN INCI¹ List | % Concentration in bulk (w/w) |
|---|---|
| Aqua | 58.0 |
| Homosalate | 10 |
| Octocrylene | 7.5 |
| Ethylhexyl Salicylate | 15 |
| Glyceryl stearate | 3.3 |
| Glycerin | 13 |
| Butyl Methoxydibenzoylmethane | 3 |
| C12-15 alcohols benzoate | 2 |
| Ethylhexyl Methoxyxinnamate | 12 |
| Cetearyl Alcohol | 1.65 |
| Phenoxyethanol | 0.9 |
| Behenin Wax | 0.75 |
| Butyrospermum Parkii | 0.75 |
| Potassium cetyl phosphate | 0.55 |
| Xantham Gum | 0.4 |
| Ethylhexyl Glycerin | 0.1 |
| Tetrasodium EDTA | 0.1 |
| Citric Acid | 0.1 |

¹INCI: International Nomenclature Cosmetic Ingredients.

Table 2 showing the composition of an exemplary organic sunscreen composition (“Organic Sunscreen B”).

Where present in the following examples (and particularly, Examples 1 and 2), ergosterol at 0.01% (w/w) was dissolved in a small volume of ethanol and stirred into the bulk formulations during cooling.

### Example 1—Ergosterol-Containing Formulations

The exemplary sunscreens (as described above) were diluted 1:2 with Tris buffered saline pH 7.4 @37° C. and 1.0 ml of diluted sunscreen was suspended in a quartz cuvette maintained at 37° C. A xenon arc lamp (Model 66021, Thermo Oriel) fitted with a Broadband UVB 290-310 nm filter (300FS10-50 filter, L.O.T Oriel) and fibre optic guide was used as light source. The contents of the cuvette were irradiated for 1.5 hrs, total energy 15.12 J/cm². 50 μl aliquots of the samples were taken, diluted appropriately and used to determine Vitamin D₂ (Ergocalciferol) using a Vitamin D₂ ELISA kit (Abbexa Ltd, Cambridge UK. Catalog Number abx575377). The results are shown in Table 3.

Ergocalciferol (ng/ml)

| SUNSCREEN | Without radiation | After radiation |
|---|---|---|
| Physical Sunscreen A | n.d.¹ | n.d. |
| Organic Sunscreen B | n.d. | n.d. |
| Physical Sunscreen A + | n.d. | 12.7 ± 1.4 |
| 0.01% Ergosterol | | |
| Organic Sunscreen B + | n.d. | 8.6 ± 1.8 |
| 0.01% Ergosterol | | |

¹n.d.: not detected

Table 3 shows the levels of ergocalciferol generated from the example compositions following exposure to UV radiation.

Following UV irradiation, levels of ergocalciferol were observed in both the exemplary physical and organic sunscreen compositions. No ergocalciferol was observed in the sunscreens in the absence of ergosterol.

It was observed that levels of ergocalciferol were higher in the physical sunscreen composition. Without being bound by theory, the inventors hypothesise that the physical sunscreen components may have a greater effect on ergocalciferol production than organic sunscreen components and these physical sunscreen components (e.g. titanium dioxide (TiO₂) and zinc oxide (ZnO)) may act to enhance the ergocalciferol production from the ergosterol.

### Example 2—Liposomal Formulations of Ergosterol

Ergosterol was incorporated into phospholipid liposomes according to the method of Xiao Quan Tian and Michael F. Holick (“A Liposomal Model That Mimics the Cutaneous Production of Vitamin D₃”, THE JOURNAL OF BIOLOGICAL CHEMISTRY Vol. 274, No. 7, Issue of February 12, pp. 4174-4179, 1999).

In the following examples, dipalmitoyl phosphatidyl choline (PC) was used as the phospholipid.

The phospholipid (dipalmitoyl phosphatidyl choline (PC)) to ergosterol molar ratio in the phospholipid bilayers was 2 mol % of ergosterol i.e. 0.135 mM ergosterol and 6.75 mM phospholipid. The phospholipid was dissolved in chloroform/methanol 2:1 at 6.75 mM and ergosterol dissolved in hexane at 0.135 mM. An equal volume of each of the stock solutions of the phospholid and ergosterol was placed in a conical glass flask and mixed. The organic solvent was evaporated under a stream of nitrogen. The resulting thin lipid film was resuspended in a 10 mM phosphate buffer (pH 7.4) by vortexing followed by sonication in a heated water bath.

The resulting liposomes (lipid concentration 6.75 mM; ergosterol 0.135 mM, molar ratio of lipid to ergosterol, 100:2) were added to the sunscreen formulations to give a final concentration of ergosterol of 0.01% w/w.

The results are shown in Table 4.

Ergocalciferol (ng/ml)

| SUNSCREEN | Without radiation | After radiation |
|---|---|---|
| Physical Sunscreen + | n.d.¹ | 74.3 ± 18.6 |
| 0.01% Liposomal | | |
| Ergosterol | | |
| Organic Sunscreen + | n.d | 84.6 ± 21.3 |
| 0.01% Liposomal | | |
| Ergosterol | | |

¹n.d.: not detected

Table 4 shows the levels of ergocalciferol generated from the example compositions following exposure to UV radiation.

Following UV irradiation, surprisingly it was observed that the liposomal formulations of ergosterol significantly increased the levels of ergocalciferol detected in both the exemplary physical and organic sunscreen compositions following UV radiation.

### Example 3—Ergosterol-, Thiol- and Nitrate-Supplemented Formulations

### Example 3a

Sodium nitrate, glutathione and ergosterol were mixed into the exemplary physical sunscreen A by stirring aqueous solutions (of glutathione and nitrate) or ethanolic solutions (of ergosterol) into the formulation. Samples were diluted 1:1 with Tris buffered saline pH 7.4 @37° C. and 1.0 ml of diluted sunscreen was suspended in a quartz cuvette maintained at 37° C. A xenon arc lamp (Model 66021, Thermo Oriel) fitted with a Broadband UVB (260-320 nm) filter and fibre optic guide was used as light source. The contents of the cuvette were irradiated for 1.5 hrs, total energy 15.12 J/cm². 500 μl aliquots of the samples were taken and stored at room temperature for 24 hours. Vitamin D₂ (Ergocalciferol) production was determined using a Vitamin D₂ ELISA kit (Abbexa Ltd. Cambridge UK. Catalog Number abx575377).

The results are shown in Table 5 below.

Ergocalciferol (Vitamin D₂) ng/ml)

| SUNSCREEN | Without radiation | After radiation |
|---|---|---|
| Physical Sunscreen A | n.d. | n.d. |
| Physical Sunscreen A + | n.d. | 11.5 ± 1.1 |
| 0.01% w/w Ergosterol | | |
| Physical Sunscreen A + | n.d | 15.7 ± 2.9* |
| 0.01% w/w Ergosterol + | | |
| 0.1% w/w Sodium Nitrate | | |
| Physical Sunscreen A + | n.d. | 32.3 ± 14.6* |
| 0.01% w/w Ergosterol + | | |
| 0.1% w/w Glutathione | | |
| Physical Sunscreen A + | n.d. | 58.2 ± 12.3* |
| 0.01% w/w Ergosterol + | | |
| 0.1% w/w Sodium Nitrate + | | |
| 0.1% w/w Glutathione | | |

Table 5 shows the effects of glutathione and sodium nitrate on the production of Ergocalciferol (Vitamin D₂) in an exemplary physical sunscreen formulation A. Results represent the mean±S.D of three independent experiments. *p<0.01 vs ergosterol alone. % w/w amounts in the table above indicate a percentage by weight of the compound based on the total weight of the sunscreen composition. n.d.=not detected.

### Example 3b

Sodium nitrate, glutathione and ergosterol were mixed into the exemplary organic sunscreen B by stirring aqueous solutions (glutathione and nitrate) or ethanolic solutions (ergosterol) into the formulation. Samples were diluted 1:1 with Tris buffered saline pH 7.4 @37° C. and 1.0 ml of diluted sunscreen was suspended in a quartz cuvette maintained at 37° C. A xenon arc lamp (Model 66021, Thermo Oriel) fitted with a Broadband UVB (260-320 nm) filter and fibre optic guide was used as light source. The contents of the cuvette were irradiated for 1.5 hrs, total energy 15.12 J/cm². 500 μl aliquots of the samples were taken and stored at room temperature for 24 hours. Vitamin D₂ (Ergocalciferol) production was determined using a Vitamin D₂ ELISA kit (Abbexa Ltd. Cambridge UK. Catalog Number abx575377).

The results are shown in Table 6.

Ergocalciferol (Vitamin D₂) ng/ml)

| SUNSCREEN | Without radiation | After radiation |
|---|---|---|
| Organic Sunscreen B | n.d.¹ | n.d. |
| Organic Sunscreen B + | n.d. | 10.6 ± 1.1 |
| 0.01% w/w Ergosterol | | |
| Organic Sunscreen B + | n.d | 14.8 ± 2.1* |
| 0.01% w/w Ergosterol + | | |
| 0.1% w/w Sodium Nitrate | | |
| Organic Sunscreen B + | n.d. | 31.4 ± 9.5* |
| 0.01% w/w Ergosterol + | | |
| 0.1% w/w Glutathione | | |
| Organic Sunscreen B + | n.d. | 68.9 ± 12.7* |
| 0.01% w/w Ergosterol + | | |
| 0.1% w/w Sodium Nitrate + | | |
| 0.1% w/w Glutathione | | |

Table 6 shows the effects of glutathione and sodium nitrate (“SunVitamin+”) on the production of PreVitamin D₂ in an exemplary organic sunscreen formulation B. Results represent the mean±S.D of three independent experiments. % w/w amounts in the table above indicate a percentage by weight of the compound based on the total weight of the sunscreen composition. n.d.=not detected.

The results shown in tables 5 and 6 indicate that a thiol (such as glutathione) and/or a metal nitrate (such as sodium nitrate) can be used to enhance radiation-induced production of ergocalciferol from ergosterol in both physical and organic sunscreens.

### Example 4

Sodium nitrate, glutathione and ergosterol (sometimes referred to herein as “SunVitamin+” ingredients) were mixed into the exemplary sunscreens A and B by stirring aqueous solutions (of glutathione and sodium nitrate) or ethanolic solution (of ergosterol) into the formulations. Samples were diluted 1:1 with Tris buffered saline pH 7.4 @37° C. and 1.0 ml of diluted sunscreen was suspended in a quartz cuvette maintained at 37° C. A xenon arc lamp (Model 66021, Thermo Oriel) fitted with a Broadband filter and fibre optic guide was used as light source. The contents of the cuvette were irradiated for 1.0 hr, total energy 10.08 J/cm². Triplicate 50 μl aliquots of the irradiated samples were taken and placed in a 96 well flat bottom plate for determination of NO derived nitrite using the Promega Griess Reagent System (Product G2930). 50 μl of the stock Promega Sulphanilamide solution (1% sulphanilamide in 5% phosphoric acid) was added to all experimental samples and wells containing a dilution series for the Nitrite standard reference curve. Samples were incubated for 5-10 minutes at room temperature, protected from light. 50 μl of the Promega NED (0.1% N-1-napthylethylenediamine dihydrochloride in water) solution was then added to all wells and the plate incubated at room temperature for 5-10 minutes, protected from light. Absorbance was measured within 30 minutes in a plate reader with a filter between 520 nm and 550 nm.

The results are shown in Table 7 below.

| | PHYSICAL | ORGANIC |
|---|---|---|
| | SUNSCREEN A | SUNSCREEN B |
| | Nitric Oxide Production pmoles/50 μl | |
| +0.01% w/w Ergosterol + | n.d | n.d. |
| 0.1% w/w sodium nitrate + | 21 ± 8 | 18 ± 4.4 |
| 0.1% w/w Glutathione + | n.d. | n.d. |
| 0.1% w/w sodium nitrate + | 423 ± 37 | 548 ± 51 |
| 0.1% w/w Glutathione + | | |
| 0.1% w/w sodium nitrate + | 763 ± 96 | 826 ± 77 |
| 0.1% w/w Glutathione + | | |
| 0.01% w/w Ergosterol | | |

Table 7 shows the effects of Ergosterol on the UV induced production of nitric oxide by sodium nitrate and glutathione (“SunVitamin+” ingredients) in exemplary sunscreen formulations. % w/w amounts in the table above indicate a percentage by weight of the compound based on the total weight of the sunscreen composition. n.d.=not detected.

The results shown in Table 7 indicate that the presence of Ergosterol can also unexpectedly enhance UV induced NO production from glutathione and nitrate.

### Example 5

Sodium nitrate, glutathione and ergosterol (sometimes referred to herein as “SunVitamin+” ingredients) were mixed into the exemplary sunscreens A and B in accordance with the method outlined in example 4. For those example formulations comprising dipalmitoylphosphatidylcholine (DPPC), ergosterol was incorporated into the phospholipid liposomes in accordance with the method outlined in example 2. Ergocalciferol production and nitric oxide production were determined in accordance with the method outlined in example 4.

The results are shown in Table 8 below.

| | | Nitric Oxide |
|---|---|---|
| | Ergocalciferol | Production |
| | Production ng/ml | pmoles/50 μl |
| Physical Sunscreen A + | 57 ± 7 | 697 ± 52 |
| 0.1% w/w sodium nitrate + | | |
| 0.1% w/w Glutathione + | | |
| 0.01% w/w Ergosterol | | |
| Physical Sunscreen A + | 119 ± 21 | 925 ± 47 |
| 0.1% w/w sodium nitrate + | | |
| 0.1% w/w Glutathione + | | |
| 0.01% w/w Ergosterol + | | |
| 0.1% w/w DPPC Liposomes | | |
| Organic Sunscreen B + | 82 ± 11 | 769 ± 33 |
| 0.1% w/w sodium nitrate + | | |
| 0.1% w/w Glutathione + | | |
| 0.01% w/w Ergosterol | | |
| Organic Sunscreen B + | 147 ± 18 | 992 ± 73 |
| 0.1% w/w sodium nitrate + | | |
| 0.1% w/w Glutathione + | | |
| 0.01% w/w Ergosterol + | | |
| 0.1 wt % DPPC Liposomes | | |

Table 8 shows the effects of dipalmitoylphosphatidylcholine (DPPC) liposomes on UV induced production of nitric oxide and ergocalciferol from exemplary sunscreen formulations A and B containing sodium nitrate, glutathione and ergosterol. % w/w amounts in the table above indicate a percentage by weight of the compound based on the total weight of the sunscreen composition.

Following UV irradiation, it was observed that the liposomal formulations of ergosterol significantly increased the levels of ergocalciferol detected in both the exemplary physical and organic sunscreen compositions following UV radiation. Additionally, an enhancement in NO production was observed.

Although the present invention has been described in some detail by way of illustration and example for purposes of clarity and understanding, the description and examples should not be construed as limiting the scope of the invention. The disclosures of all patent and scientific literature herein are expressly incorporated in their entirety by reference.

1. A composition for use in facilitating, augmenting and/or promoting the generation of vitamin D₂, wherein the composition:
(i) comprises ergosterol; and
(ii) is formulated for topical administration.

2. A composition comprising:
(i) one or more UV-protecting and/or sunlight-protecting components; and
(ii) ergosterol.

3. The composition of claim 2, wherein the one or more UV-protecting and/or sunlight-protecting components are selected from organic chemicals, organic particulates, inorganic chemicals, inorganic particulates, or combinations thereof.

4. The composition of any one of the preceding claims, further comprising:
(iii) one or more promoter agents to augment, promote and/or enhance the generation of vitamin D₂.

5. The composition of any one of the preceding claims, wherein the one or more promoter agents is selected from a thiol compound, a disulfide compound, an NO-precursor compound and a liposome, such as a phospholipid.

6. The composition of claim 5, wherein the one or more promoter agents comprises an NO-precursor compound selected from nitrite-containing compounds, nitrate-containing compounds and nitro-containing organic compounds, optionally wherein the NO-precursor compound is a metal nitrate.

7. The composition of claim 5 or 6, wherein the one or more promoter agents comprises: (i) a thiol compound selected from glutathione, cysteine, homocysteine, cysteamine, and thiolactate; or (ii) a disulfide compound selected from dithioglycolate, lipoic acid (oxidised) and cystine.

8. The composition of any one of claims 5 to 7, wherein the one or more promoter agent is a phospholipid, optionally wherein the phospholipid is or comprises a phosphatidylcholine.

9. The composition of claim 8, wherein the molar ratio of the ergosterol to the phospholipid is between about 1:10 and 1:200, or between about 1:25 and 1:100.

10. The composition of any one of claims 4 to 9, wherein the promoter agent provides at least a two-fold increase in the amount of vitamin D₂ produced from the composition when compared to the amount of vitamin D₂ produced from the composition in the absence of the promoter agent.

11. A composition comprising:
(i) ergosterol;
(ii) a thiol and/or a disulfide compound (e.g. glutathione); and
(iii) a NO-precursor compound (e.g. a metal nitrate such as sodium nitrate);
optionally wherein the composition further comprises:
(iv) a liposome (e.g. a phospholipid); and/or
(v) one or more UV-protecting and/or sunlight-protecting components.

12. The composition of any one of the preceding claims, wherein the composition comprises between about 0.0001 wt % and about 25 wt %, or between about 0.0075 wt % and about 1 wt % or about 0.1 wt % of ergosterol based on the total weight of the composition.

13. The composition of any one of the preceding claims, wherein an NO-precursor compound is present and the composition comprises between about 0.01 wt % and about 1 wt %, between about 0.05 wt % and about 0.5 wt %, or between about 0.075 wt % and about 0.2 wt % of the NO-precursor compound based on the total weight of the composition.

14. The composition of any one of the preceding claims, wherein a thiol or disulfide compound is present and the composition comprises between about 0.01 wt % and about 1 wt %, between about 0.05 wt % and about 0.5 wt %, or between about 0.075 wt % and about 0.2 wt % of the thiol or disulfide compound based on the total weight of the composition.

15. The composition of any one of the preceding claims, which is formulated for topical administration.

16. The composition of any one of the preceding claims, further comprising one or more pharmaceutically, therapeutically and/or cosmetically acceptable diluents, excipients or carriers.

17. Ergosterol for use in a method for treating and/or preventing diseases or conditions associated with reduced levels of vitamin D and/or a vitamin D deficiency, said method comprising topically administering to a subject in need thereof a therapeutically effective amount of ergosterol.

18. A method for augmenting levels of vitamin D (e.g. vitamin D₂) in a subject in need thereof, said method comprising using, applying and/or administering the composition of any one of claims 1 to 16 to the subject in need thereof.

19. A composition for use in a method for treating and/or preventing diseases or conditions associated with reduced levels of vitamin D and/or a vitamin D deficiency, said method comprising administering to a subject in need thereof a therapeutically effective amount of the composition of any one of claims 1 to 16.

20. A method for treating and/or preventing diseases or conditions associated with reduced levels of vitamin D (e.g. vitamin D₂) and/or a vitamin D (e.g. vitamin D₂) deficiency, said method comprising:
(i) administering to a subject in need thereof a therapeutically effective amount of the composition of any one of claims 1 to 16; or
(ii) topically administering to a subject in need thereof a therapeutically effective amount of ergosterol.

21. Use of ergosterol or a composition of any one of claims 1 to 16 in the manufacture of a medicament for use in:
(i) the treatment and/or prevention of diseases or conditions associated with reduced levels of vitamin D and/or a vitamin D deficiency; and/or
(ii) a method for increasing and/or augmenting levels of vitamin D (e.g. vitamin D₂) in a subject in need thereof.

22. The ergosterol for use of claim 17, the composition for use of claim 19, the method of claim 18 or 20, or the use of claim 21, wherein the diseases or conditions associated with reduced levels of vitamin D and/or a vitamin D deficiency are selected from diseases of the skeletomuscular system (e.g. bone diseases such as Rickets, osteomalacia and osteoporosis), multiple sclerosis (MS), Alzheimer's Disease, cognitive impairment, asthma, cardiovascular disease, seasonal disorders, inflammation, depression diabetes, and cancer.

23. A method for increasing and/or augmenting levels of nitric oxide in a subject in need thereof, said method comprising using, applying and/or administering to the subject a composition according to any one of claims 11 to 16.

24. A method for treating and/or preventing diseases or conditions associated with reduced levels of nitric oxide and/or nitric oxide deficiency, said method comprising administering to a subject in need thereof a therapeutically effective amount of a composition according to any one of claims 11 to 16.

25. A composition according to any one of claims 11 to 16 for use in a method for treating and/or preventing diseases or conditions associated with reduced levels of nitric oxide and/or a nitric oxide deficiency.

26. Use of a composition according to any one of claims 11 to 16, in the manufacture of a medicament for use in:
(i) the treatment and/or prevention of diseases or conditions associated with reduced levels of nitric oxide and/or a nitric oxide deficiency; and/or
(ii) a method for increasing and/or augmenting levels of nitric oxide in a subject in need thereof.

27. The ergosterol for use of claim 17 or 22, the composition for use of claim 19, 22 or 25, the method of claim 18, 20, 22, 23 or 24, or the use of claim 21, 22 or 26, wherein the treatment is a prophylactic treatment.

28. The ergosterol for use of claim 17, 22 or 27, the composition for use of claim 19, 22, 25 or 27, the method of claim 18, 20, 22, 23, 24, or 27, or the use of claim 21, 22, 26 or 27, wherein the composition is applied topically to the subject in need thereof.

29. The ergosterol for use of claim 17, 22, 27 or 28, the composition for use of claim 19, 22, 25, 27 or 28, the method of claim 18, 20, 22, 23, 24, 27 or 28, or the use of claim 21, 22, 26, 27 or 28, wherein the composition is administered, applied or used by a subject:
(i) who is to be exposed to the sun; and/or
(ii) a subject susceptible, predisposed to or at risk of developing a disease or condition associated with reduced levels of vitamin D and/or a vitamin D deficiency, and optionally a disease or condition associated with reduced levels of nitric oxide and/or a nitric oxide deficiency.

30. A method for preparing a composition according to any one of claims 1 to 16.

31. The method of claim 30, further comprising adding ergosterol to one or more UV-protecting and/or sunlight-protecting components to provide the composition.

32. The method of claim 30 or 31, further comprising adding:
(i) one or more promoter agents to augment, promote and/or enhance the generation of vitamin D₂; and/or
(ii) a hydrogel to the composition.

33. The method of claim 32, wherein the ergosterol and the one or more promoter agents (e.g. liposomes) may be formulated together prior to addition to the composition.
